(19) United States
(12) Patent Application Publication
(10) Pub. No.: US 2008/0161409 A1
(43) Pub. Date: Jul. 3, 2008

(54) COMPOSITIONS FOR TREATEMENT OF CROUP AND METHODS OF ADMINISTERING SAME

(76) Inventors: W. Randolph WARNER, Punta Gorda, FL (US); Werner Gutmann, Powhatan, VA (US)

Correspondence Address: MCGUIREWOODS, LLP, 1750 TYSONS BLVD, SUITE 1800, MCLEAN, VA, 22102, US

(21) Appl. No.: 11/935,291

(22) Filed: Nov. 5, 2007

Related U.S. Application Data
(60) Provisional application No. 60/864,261, filed on Nov. 3, 2006.

(51) Int. Cl. A61K 31/137; A61P 11/00

(52) U.S. Cl. 514/649

(57) ABSTRACT

The invention relates to a racemic epinephrine inhalation solution, system, kit, and method for the treatment of croup. In particular, the racemic epinephrine solution is premixed, sterile, premeasured single unit dose of racemic epinephrine for the treatment of croup. More particularly, the racemic inhalation solution is free of antimicrobial preservatives and other preservatives. Additionally, the invention also relates to a comprehensive kit to administer racemic epinephrine aerosol therapy for the treatment of croup. More particularly, the invention relates to an inhalation therapy kit to provide a seamless conduit for a patient to receive in-office or hospital inhalation treatments and access to drugs and equipment for continuing home use.

### CROSS-REFERENCE TO RELATED APPLICATIONS

This application claims priority under 35 U.S.C. §119(e) to provisional U.S. Patent Application No. 60/864,261, filed on Nov. 3, 2006, the disclosure of which is expressly incorporated by reference herein in its entirety.

### FIELD OF THE INVENTION

The invention relates to a racemic epinephrine inhalation solution, system, kit, and method for the treatment of croup. In particular, the racemic epinephrine solution is premixed, sterile, premeasured single unit dose of racemic epinephrine for the treatment of croup. More particularly, the racemic inhalation solution is free of antimicrobial preservatives and other preservatives.

### BACKGROUND OF THE INVENTION

Croup, also referred to as croup syndrome or laryngotracheobronchitis, is an acute inflammation of the upper and lower respiratory tracts. Croup commonly afflicts infants and young children, typically aged between 3 months and 5 years. It is characterized by a barking cough and inspiratory stridor. The “barking” cough of croup is diagnostic. Stridor, which is a high-pitched sound heard on inhalation, will be provoked or worsened by agitation of crying. If stridor is heard when the child is calm, critical narrowing of the airway may be imminent.

The parainfluenza viruses, and particularly parainfluenza virus type I and type III, are the major pathogens. Less common pathogens include respiratory syncytial virus (RSV), influenza A and B viruses, adenovirus, enterovirus, rhinovirus and measles virus and Mycoplasmapneumoniae. The inflammatory response to the infection causes the respiratory distress, and not the infection itself. In general, it usually afflicts young children because their airways are smaller and differently shaped than adults, making them more susceptible.

The treatment of croup depends upon the severity of symptoms. The current cornerstones of treatment are glucocorticoids and nebulized epinephrine. While steroids have proven beneficial in mild-to-severe illness, epinephrine is typically reserved for patients in moderate-to-severe distress. Although a child who is sufficiently symptomatic to receive epinephrine may be discharged after 3-4 hours of observation, anyone receiving epinephrine should also be given steroids.

In general, epinephrine, racemic (microNefrin) 2.25% is administered as a mixture of dextro and levo isomers. Racemic epinephrine causes adrenergic stimulation, which constricts precapillary arterioles, thus decreasing capillary hydrostatic pressure. This leads to fluid resorption from the interstitium and improvement in the laryngeal mucosal edema, although its β2 activity leads to bronchial smooth muscle relaxation.

Currently, racemic epinephrine is configured as a 2.25% solution in a 0.5 ml solution in a 0.5 ml vial. In order to administer treatment in a jet nebulizer, the 0.5 ml dose has to be diluted to 3 ml with 2.5 ml of normal saline. There are several disadvantages associated with the current drug configuration and administration practice. First, parents, care givers and other typically do not have adequate experience diluting this medication, resulting in contamination or inappropriate dosing, among other problems. Secondly, antimicrobial preservatives and other preservatives are present in the currently available racemic epinephrine inhalation solution. For example, the current epinephrine inhalation solution contains sodium metabisulfite and potassium metabisulfite. Sulfites may cause allergic-type reactions including anaphylactic symptoms and life-threatening or less severe asthmatic episodes in certain susceptible people.

Therefore, there is a need for an improved epinephrine inhalation solution, system, kit and method for the treatment of croup.

### SUMMARY OF THE INVENTION

The invention satisfies the above needs by providing an improved racemic epinephrine inhalation solution, comprehensive kit and methods for caregivers to administer aerosol therapy to a patient in need.

One aspect of the invention is to provide a racemic epinephrine solution for the treatment of croup. Another aspect is to provide a prepackaged, sterile, premixed, premeasured, sterile, racemic epinephrine inhalation solution for the treatment of croup.

A further aspect of the invention is to provide a preservative-free racemic epinephrine solution for the treatment of croup.

According to an aspect of the invention, an aerosolized therapy kit is provided which comprises a thermoformed tray, a nebulizer, tubing, a pediatric mask, at least one vial containing an effective amount of the sterile, premixed, premixed racemic epinephrine to be administered, and at least one preprinted prescription.

### BRIEF DESCRIPTION OF THE DRAWINGS

The accompanying drawing, which is included to provide a further understanding of the invention, is incorporated in and constitutes a part of this specification, illustrates embodiments of the invention, and together with the detailed description, serves to explain the principles of the invention. No attempt is made to show structural details of the invention in more detail than may be necessary for a fundamental understanding of the invention and various ways in which it may be practiced.

FIG. 1 shows an aerosol therapy kit of the invention, according to principles of the invention.

### DETAILED DESCRIPTION OF THE INVENTION

It is understood that the invention is not limited to the particular methodology, protocols, and reagents, etc., described herein, as these may vary as the skilled artisan will recognize. It is also to be understood that the terminology used herein is used for the purpose of describing particular embodiments only, and is not intended to limit the scope of the invention. It also is be noted that as used herein and in the appended claims, the singular forms “a,” “an,” and “the” include the plural reference unless the context clearly dictates otherwise. Thus, for example, a reference to “a preservative” is a reference to one or more preservatives and equivalents thereof known to those skilled in the art.

Unless defined otherwise, all technical and scientific terms used herein have the same meanings as commonly understood by one of ordinary skill in the art to which the invention pertains. The embodiments of the invention and the various features and advantageous details thereof are explained more fully with reference to the non-limiting embodiments and/or illustrated in the accompanying drawings and detailed in the following description. It should be noted that the features illustrated in the drawings are not necessarily drawn to scale, and features of one embodiment may be employed with other embodiments as the skilled artisan would recognize, even if not explicitly stated herein.

Moreover, provided immediately below is a “Definition” section, where certain terms related to the invention are defined specifically. Particular methods, devices, and materials are described, although any methods and materials similar or equivalent to those described herein can be used in the practice or testing of the invention. All references referred to herein are incorporated by reference herein in their entirety.

### DEFINITIONS

The terms “active agent,” “drug” and “pharmacologically active agent” are used interchangeably herein to refer to a chemical material or compound which, when administered to an organism (human or animal) induces a desired pharmacologic effect. Included are derivatives and analogs of those compounds or classes of compounds specifically mentioned that also induce the desired pharmacologic effect.

By the terms “effective amount” or “therapeutically effective amount” of an agent as provided herein are meant a nontoxic but sufficient amount of the agent to provide the desired therapeutic effect. The exact amount required will vary from subject to subject, depending on the age, weight, and general condition of the subject, the severity of the condition being treated, the judgment of the clinician, and the like. Thus, it is not possible to specify an exact “effective amount.” However, an appropriate “effective” amount in any individual case may be determined by one of ordinary skill in the art using only routine experimentation.

The terms “treating” and “treatment” as used herein refer to reduction in severity and/or frequency of symptoms, elimination of symptoms and/or underlying cause, prevention of the occurrence of symptoms and/or their underlying cause, and improvement or remediation of damage. Thus, for example, the present method of “treating” croup, as the term “treating” is used herein, encompasses treatment of croup in a clinically symptomatic individual.

The terms “condition,” “disease” and “disorder” are used interchangeably herein as referring to a physiological state that can be prevented or treated by administration of a pharmaceutical formulation as described herein. Exemplary diseases and conditions may include, but not limited to, asthma, acute lower respiratory tract infection, chronic obstructive pulmonary disease, acute bronchitis, bronchiectasis, environmental pulmonary diseases, pulmonary hypertension, mediastinal and pleural disorders, pneumonia, infant respiratory distress syndrome, croup, bronchitis, and pertussis.

The term “patient” as in treatment of “a patient” refers to a mammalian individual afflicted with or prone to a condition, disease or disorder as specified herein, and includes humans, adults and children, and animals.

The invention relies on the therapeutic effects of epinephrine for the treatment of croup. As used herein, the term “epinephrine” includes, without limitation, any form of epinephrine which is capable of producing the desired effect in patients, and in particular, pediatric patients, including but not limited to, all tautomeric forms, enantiomeric forms, stereoisomers, anhydrides, acid addition salts, base salts, solvates, analogues, and derivatives of racemic epinephrine. Acceptable salts of epinephrine may include hydrochloride, sulfate, maleate, tartrate, citrate, and the like.

According to an embodiment of the invention, epinephrine has the empirical formula C₉H₁₃NO₃. The chemical name for epinephrine is 4-(1-hydroxy-2-(methylamino)ethyl)benzene-1,2-diol, and its established chemical structure is as follows:

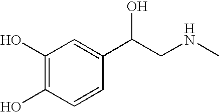

According to one embodiment of the invention, the epinephrine may be provided in a variety of pharmaceutically acceptable vehicles, including without limitation, water, saline, or other aqueous solutions including a pharmaceutically acceptable amount of an osmotic agent.

In a further embodiment, the inhalation solution of the invention may comprise a therapeutically effective pediatric amount of epinephrine. For example, the therapeutically effective pediatric amount of epinephrine may include from about 2.25%, which includes 1.125% d-epinephrine and 1.125% l-epinephrine.

Currently, the epinephrine inhalation solution contains antimicrobial preservatives and other types of preservatives. In particular, the epinephrine solution contains sodium metabisulfite and potassium metabisulfite. The inhalation solution of the invention may be provided without preservatives, thereby making it more suitable for pediatric patients.

In one embodiment, the inhalation solution of the invention may be provided in sterile, unit dose treatments, thereby eliminating the need for preservatives in the solution. Another advantage of a sterile, premixed, premeasured inhalation solution is that it reduces the possibility of introducing contaminants into the patient when administered, thus reducing the risk of an opportunistic infection in the patient. Additionally, providing the inhalation solution in such a manner eliminates the need to dilute the epinephrine solution to obtain proper dosages for treatment.

According to one embodiment, the inhalation solution of the invention may be packaged in blow-filled ampoules. The blow-filled ampoules may be prefilled with 3 ml of solution which includes 2.25% of the active drug, such as racemic epinephrine, pre-mixed in sterile 0.9% saline. Moreover, the inhalation solution packaged in the blow-filled ampoule would be preservative-free.

In one embodiment, the inhalation solution of the invention may be administered by nebulizer such as jet nebulizer, ultrasonic nebulizer, and breath-enhanced nebulizer. In a specific embodiment, the inhalation solution is administered in a jet nebulizer.

The invention relates to a therapy kit to provide healthcare givers a comprehensive unit to provide aerosolized drug treatments to a patient. More particularly, the aerosolized therapy kits of the invention may be comprehensive kits for the treatment of pulmonary diseases or pulmonary infection, such as croup.

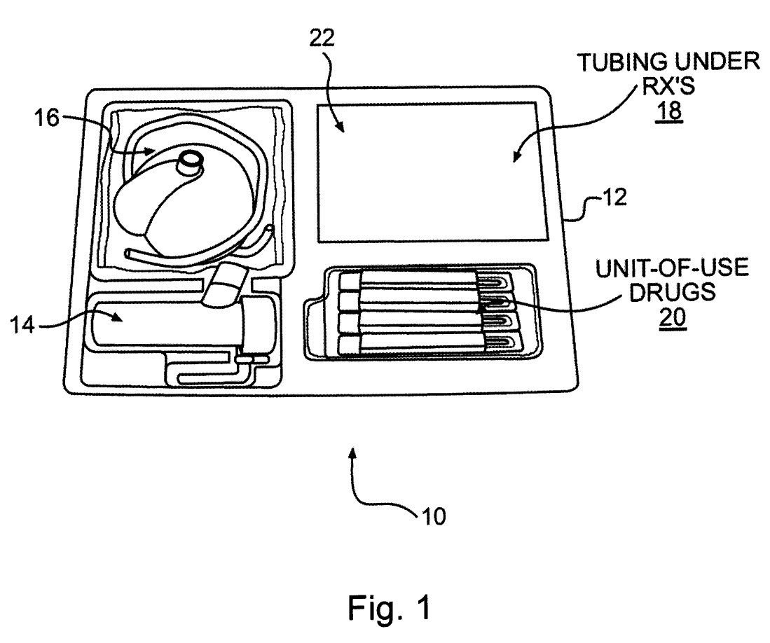

The aerosol therapy kit may contain all items that are needed for a caregiver to administer inhalation aerosol therapy to a patient. For example, in general, the aerosol therapy kit may contain at least one nebulizer, at least one mask, tubing, at least one drug to be administered to the patient, and at least one pre-filled prescription form for follow-up or preventive treatment. FIG. 1, which shows an embodiment of the invention, shows an aerosol therapy kit 10 containing a thermoform tray 12, a nebulizer 14, a nebulizer mask 16, tubing 18, unit-of-use drug 20, and pre-filled or pre-printed prescriptions 22.

The contents of each kit would depend upon the type of treatment to be administered to a patient. According to another embodiment, the aerosol therapy kit may be used for the treatment of croup. Here, the aerosol kit of the invention may contain a nebulizer, tubing, a mask appropriate for use with a pediatric patient, such as an infant or child, 3 ml vial of 2.25% racemic epinephrine diluted in 0.9% saline that is preservative-free, a 5 ml unit-dose vial of liquid oral predniosolone sodium phosphate (15 mg/ml), and pre-printed prescriptions for PARI Pred-Pack. In a further embodiment, the aerosol kit may also contain a nebulizer compressor system.

In an alternative embodiment, the aerosol therapy kits of the invention may be assembled or modified to be suitable for treatment of any pulmonary disorders and diseases. For example, the aerosol therapy kits may be used for the treatment of asthma, acute lower respiratory tract infection, chronic obstructive pulmonary disease, acute bronchitis, bronchiectasis, environmental pulmonary diseases, pulmonary hypertension, mediastinal and pleural disorders, pneumonia, infant respiratory distress syndrome, croup, bronchitis, and pertussis. For example, an aerosol therapy kit may be assembled which includes an effective amount of a drug, such as short-acting bronchodilator/beta agonist pulmicort respules, budesonide inhalation suspension, corticosteroid/anti-inflammatory racepinephrine, and (racemic epinephrine) bronchodilator.

Further exemplary drug compounds for use in the aerosol therapy kit of the invention, may include but not limited to, alprazolam; amoxapine; atropine; bumetanide; buprenorphine; butorphanol; clomipramine; donepezil; hydromorphone; loxapine; midazolam; morphine; nalbuphine; naratriptan; olanzapine; paroxetine; pramipexole; prochlorperazine; quetiapine; rizatriptan; sertraline; sibutramine; sildenafil; sumatriptan; tadalafil; vardenafil; venlafaxine; zolpidem; apomorphine HCl; celecoxib; ciclesonide; eletriptan; parecoxib; valdecoxib; fentanyl; citalopram; escitalopram; clonazepam; oxymorphone; albuterol; sufentanyl; and remifentanyl.

Additionally, the drug vials provided in the therapy kit may contain an effective dose of, for example, substances selected from the groups of anti-inflammatory compounds, anti-allergics, glucocorticoids, anti-infective agents, antibiotics, antifungals, antivirals, mucolytics, antiseptics, vasoconstrictors, wound healing agents, local anaesthetics, peptides, and proteins.

Examples of potentially useful anti-inflammatory compounds that may be applicable for aerosol therapy may include glucocorticoids and non-steroidal anti-inflammatory agents such as betamethasone, beclomethasone, budesonide, ciclesonide, dexamethasone, desoxymethasone, fluoconolone acetonide, flucinonide, flunisolide, fluticasone, icomethasone, rofleponide, triamcinolone acetonide, fluocortin butyl, hydrocortisone, hydroxycortisone-17-butyrate, prednicarbate, 6-methylprednisolone aceponate, mometasone furoate, elastane, prostaglandin, leukotriene, bradykinin antagonists, non-steroidal anti-inflammatory drugs (NSAIDs), such as ibuprofen, indometacin, including any pharmaceutically acceptable salts, esters, isomers, stereoisomers, diastereomers, epimers, solvates or other hydrates, prodrugs, derivatives, or any other chemical or physical forms of active compounds comprising the respective active moieties.

Examples of potentially useful antiallergic agents applicable for aerosol therapy may include glucocorticoids, nedocromil, cetrizin, loratidin, montelukast, roflumilast, ziluton, omalizumab, Heparinoids and other antihistamines, Azelastine, Cetirizin, Desloratadin, Ebastin, Fexofenadin, Levocetirizin, Loratadin.

Examples of anti-infective agents, whose class or therapeutic category is herein understood as comprising compounds which are effective against bacterial, fungal, and viral infections, i.e. encompassing the classes of antimicrobials, antibiotics, antifungals, antiseptics, and antivirals, that may be suitable for aerosol therapy may include penicillins, including benzylpenicillins (penicillin-G-sodium, clemizone penicillin, benzathine penicillin G), phenoxypenicillins (penicillin V, propicillin), aminobenzylpenicillins (ampicillin, amoxycillin, bacampicillin), acylaminopenicillins (azlocillin, mezlocillin, piperacillin, apalcillin), carboxypenicillins (carbenicillin, ticarcillin, temocillin), isoxazolyl penicillins (oxacillin, cloxacillin, dicloxacillin, flucloxacillin), and amidine penicillins (mecillinam); cephalosporins, including cefazolins (cefazolin, cefazedone); cefuroximes (cerufoxim, cefamdole, cefotiam), cefoxitins (cefoxitin, cefotetan, latamoxef, flomoxef), cefotaximes (cefotaxime, ceftriaxone, ceftizoxime, cefmenoxime), ceftazidimes (ceftazidime, cefpirome, cefepime), cefalexins (cefalexin, cefaclor, cefadroxil, cefradine, loracarbef, cefprozil), and cefiximes (cefixime, cefpodoxim proxetile, cefuroxime axetil, cefetamet pivoxil, cefotiam hexetil), loracarbef, cefepim, clavulanic acid/amoxicillin, Ceftobiprole; synergists, including beta-lactamase inhibitors, such as clavulanic acid, sulbactam, and tazobactam; carbapenems, including imipenem, cilastin, meropenem, doripenem, tebipenem, ertapenem, ritipenam, and biapenem; monobactams, including aztreonam; aminoglycosides, such as apramycin, gentamicin, amikacin, isepamicin, arbekacin, tobramycin, netilmicin, spectinomycin, streptomycin, capreomycin, neomycin, paromoycin, and kanamycin; macrolides, including erythromycin, clarythromycin, roxithromycin, azithromycin, dithromycin, josamycin, spiramycin and telithromycin; gyrase inhibitors or fluoroquinolones, including ciprofloxacin, gatifloxacin, norfloxacin, ofloxacin, levofloxacin, perfloxacin, lomefloxacin, fleroxacin, garenoxacin, clinafloxacin, sitafloxacin, prulifloxacin, olamufloxacin, caderofloxacin, gemifloxacin, balofloxacin, trovafloxacin, and moxifloxacin; tetracyclines, including tetracycline, oxytetracycline, rolitetracycline, minocycline, doxycycline, tigecycline and aminocycline; glycopeptides, inlcuding vancomycin, teicoplanin, ristocetin, avoparcin, oritavancin, ramoplanin, and peptide 4; polypeptides, including plectasin, dalbavancin, daptomycin, oritavancin, ramoplanin, dalbavancin, telavancin, bacitracin, tyrothricin, neomycin, kanamycin, mupirocin, paromomycin, polymyxin B and colistin; sulfonamides, including sulfadiazine, sulfamethoxazole, sulfalene, co-trimoxazole, co-trimetrol, co-trimoxazine, and co-tetraxazine; azoles, including clotrimazole, oxiconazole, miconazole, ketoconazole, itraconazole, fluconazole, metronidazole, timidazole, bifonazol, ravuconazol, posaconazol, voriconazole, and omidazole and other antifungals including flucytosin, griseofluvin, tonoftal, naftifin, terbinafin, amorolfin, ciclopiroxolamin, echinocandins, such as micafungin, caspofungin, anidulafungin; nitrofurans, including nitrofurantoin and nitrofuranzone; —polyenes, including amphotericin B, natamycin, nystatin, flucocytosine; other antibiotics, including tithromycin, lincomycin, clindamycin, oxazolindiones (linzezolids), ranbezolid, streptogramine A+B, pristinamycin alpha-A+B, Virginiamycin A+B, dalfopristin/qiunupristin (Synercid), chloramphenicol, ethambutol, pyrazinamid, terizidon, dapson, prothionamid, fosfomycin, fucidinic acid, rifampicin, isoniazid, cycloserine, terizidone, ansamycin, lysostaphin, iclaprim, mirocin B17, clerocidin, filgrastim, and pentamidine; antivirals, including acyclovir, gancyclovir, birivudin, valacyclovir, zidovudine, didanosin, thiacytidin, stavudin, lamivudin, zalcitabin, ribavirin, nevirapirin, delaviridin, trifluridin, ritonavir, saquinavir, indinavir, foscamet, amantadin, podophyllotoxin, vidarabine, tromantadine, and proteinase inhibitors; plant extracts or ingredients, such as plant extracts from chamomile, hamamelis, echinacea, calendula, papain, pelargonium, essential oils, myrtol, pinen, limonen, cineole, thymol, mentol, alpha-hederin, bisabolol, lycopodin, vitapherole; wound healing compounds including dexpantenol, allantoin, vitamins, hyaluronic acid, alpha-antitrypsin, inorganic and organic zinc salts/compounds, interferons (alpha, beta, gamma), tumor necrosis factors, cytokines, interleukins.

Examples of potentially useful mucolytics that may be useful for aerosol therapy may be DNase, P2Y2-agonists (denufosol), heparinoids, guaifenesin, acetylcysteine, carbocysteine, ambroxol, bromhexine, lecithins, myrtol, and recombinant surfactant proteins.

Examples of potentially useful local anaesthetic agent which may be suitable for aerosol therapy may include benzocaine, tetracaine, procaine, lidocaine and bupivacaine.

Examples of potentially useful antiallergic agents which may be applicable for aerosol therapy may include the glucocorticoids, cromolyn sodium, nedocromil. Examples of potentially useful peptides and proteins include antibodies against toxins produced by microorganisms, antimicrobial peptides such as cecropins, defensins, thionins, and cathelicidins.

Additionally drugs to treat pulmonary hypertension, such as prostacycline analogs, iloprost, remodulin, phosphodiesterase inhibitors, such as sildenafil, vardenafil, endothelian recector antagonists, such as bosentane, virustatics, including podophyllotoxine, vidarabine, tromantadine, zidovudine; ribavirin, may be applicable for aerosol therapy.

Also, immunomodulators may be suitable for aerosol therapy may include methotrexat, azathioprine, cyclosporine, tacrolimus, sirolimus, rapamycin, mofetil, cytotatics and metastasis inhibitors, alkylants, such as nimustine, melphanlane, carmustine, lomustine, cyclophosphosphamide, ifosfamide, trofosfamide, chlorambucil, busulfane, treosulfane, prednimustine, thiotepa; antimetabolites, e.g. cytarabine, fluorouracil, methotrexate, mercaptopurine, tioguanine; alkaloids, such as vinblastine, vincristine, vindesine; antibiotics, such as alcarubicine, bleomycine, dactinomycine, daunorubicine, doxorubicine, epirubicine, idarubicine, mitomycine, plicamycine; complexes of secondary group elements (e.g. Ti, Zr, V, Nb, Ta, Mo, W, Pt) such as carboplatinum, cis-platinum and metallocene compounds such as titanocendichloride; amsacrine, dacarbazine, estramustine, etoposide, beraprost, hydroxycarbamide, mitoxanthrone, procarbazine, temiposide; paclitaxel, iressa, zactima, poly-ADP-ribose-polymerase (PRAP) enzyme inhibitors, banoxantrone, gemcitabine, pemetrexed, bevacizumab, ranibizumab.

In a further embodiment other active ingredient that may used in aerosol therapy may include, protease inhibitors, such as alpha-anti-trypsin; antioxidants, such as tocopherols, glutathion; pituitary hormones, hypothalamic hormones, regulatory peptides and their inhibiting agents, corticotropine, tetracosactide, choriogonandotropine, urofolitropine, urogonadotropine, saomatotropine, metergoline, desmopressine, oxytocine, argipressine, ornipressine, leuproreline, triptoreline, gonadoreline, busereline, nafareline, goselerine, somatostatine; parathyroide gland hormones, calcium metabolism regulators, dihydrotachysterole, calcitonine, clodronic acid, etidronic acid; thyroid gland therapeutics; sex hormones and their inhibiting agents, anabolics, androgens, estrogens, gestagenes, antiestrogenes; anti-migraine drugs, such as proxibarbal, lisuride, methysergide, dihydroergotamine, ergotamine, clonidine, pizotifene; hypnotics, sedatives, benzodiazepines, barbiturates, cyclopyrrolones, imidazopyridines, antiepileptics, zolpidem, barbiturates, phenyloin, primidone, mesuximide, ethosuximide, sultiam, carbamazepin, valproic acid, vigabatrine; antiparkinson drugs, such as levodopa, carbidopa, benserazide, selegiline, bromocriptine, amantadine, tiapride; antiemetics, such as thiethylperazine, bromopride, domperidone, granisetrone, ondasetrone, tropisetrone, pyridoxine; analgesics, such as buprenorphine, fentanyl, morphine, codeine, hydromorphone, methadone, fenpipramide, fentanyl, piritramide, pentazocine, buprenorphine, nalbuphine, tilidine; drugs for narcosis, such as N-methylated barbiturates, thiobarbiturates, ketamine, etomidate, propofol, benzodiazepines, droperidol, haloperidol, alfentanyl, sulfentanyl; antirheumatism drugs including tumor necrosis factor-alpha, nonsteroidal antiinflammatory drugs; antidiabetic drugs, such as insulin, sulfonylurea derivatives, biguanids, glitizols, glucagon, diazoxid; cytokines, such as interleukins, interferons, tumor necrosis factor (TNF), colony stimulating factors (GM-CSF, G-CSF, M-CSF); proteins, e.g. epoetine, and peptides, e.g. parathyrin, somatomedin C; heparine, heparinoids, urokinases, streptokinases, ATP-ase, prostacycline, sexual stimulants, or genetic material.

What is claimed is:

1. A composition for the treatment of croup, said composition comprising:
about 3 ml of a solution including about 2.25% epinephrine, about 0.9% saline and with the provisio that said solution does not contain preservatives.

2. A method for the treatment of croup, said method comprising the step of:
administering to a patient at least one single dispensing container including a sterile, preservative-free, inhalation solution comprising a pharmaceutically effective dose of racemic ephinephrine.

3. The method of claim 2, wherein the inhalation solution is suitable for nebulization in a nebulizer.

4. The method of claim 2, wherein the pharmaceutically effective dose is 2.25% racemic epinephrine.

5. The method of claim 2, wherein the inhalation solution further comprises saline solution.
